(19) United States
(12) Patent Application Publication
(10) Pub. No.: US 2002/0052365 A1
(43) Pub. Date: May 2, 2002

(54) Selective anxiolytic therapeutic agents

(76) Inventors: Mohler Hanns, Meilen (CH); Uwe Rudolph, Zurich (CH)

Correspondence Address: PENNIE AND EDMONDS, 1155 AVENUE OF THE AMERICAS, NEW YORK, NY, 100362711

(21) Appl. No.: 09/972,799

(22) Filed: Oct. 5, 2001

Related U.S. Application Data
(60) Provisional application No. 60/238,189, filed on Oct. 5, 2000.

(51) Int. Cl. A61K 31/5513; G01N 33/53

(52) U.S. Cl. 514/221; 435/7.1

(57) ABSTRACT

The present invention relates to selective anxiolytic therapeutic agents which allow for the treatment of anxiety-related disorders with less severe side-effects, such as sedative and amnesic effects, and in particular, dependence liability. These selective agents selectively or preferentially bind the a2-GABA_{A} receptor, as compared to the α1-GABA_{A} receptor. Alternatively, these selective agents selectively or preferentially activate the α2-GABA_{A} receptor, as compared to the α1-GABA_{A} receptor. The present invention also relates to methods for identifying such selective anxiolytic therapeutic agents. The present invention also relates to methods for identifying a molecule that decreases binding of a benzodiazepine to the α1-GABA_{A} receptor, but not substantially to the α2-GABA_{A} receptor.

The present application claims priority to U.S. Provisional Application Serial No. 60/238,189 filed Oct. 5, 2000 entitled Selective Anxiolytic Therapeutic Agents, the disclosure of which is incorporated herein by reference in its entirety.

### 1. FIELD OF THE INVENTION

The present invention relates to selective anxiolytic therapeutic agents which allow for the treatment of anxiety-related disorders with less severe side-effects, such as sedative and amnesic effects, and in particular, dependence liability. These selective agents selectively or preferentially bind the α2-GABA_{A} receptor, as compared to the α1-GABA_{A} receptor. Alternatively, these selective agents selectively or preferentially activate the α2-GABA_{A} receptor, as compared to the α1-GABA_{A} receptor. The present invention also relates to methods for identifying such selective anxiolytic therapeutic agents. The present invention also relates to methods for identifying a molecule that decreases binding of a benzodiazepine to the α1-GABA_{A} receptor, but not substantially to the α2-GABA_{A} receptor.

### 2. BACKGROUND OF THE INVENTION

Anxiety-related impairments are frequent medical conditions, and include generalized anxiety disorders, panic anxiety, posttraumatic stress disorder, phobias, anxious depression, anxiety associated with schizophrenia, restlessness and general excitation states. The main anxiolytic drugs used at present are benzodiazepines. In addition to their tranquillizing action, benzodiazepines also exert a variety of unwanted side effects, including sedative, anterograde amnesia and ethanol potentiation. However, the major factor limiting the therapeutic use of benzodiazepines are sequelae following their chronic use, in particular dependence liability. For a general review of benzodiazepines and their use in the treatment of anxiety-related disorders, see, e.g., Schader and Greenblatt, 1993, N. Engl. J. Med. 328(19):1398-1405.

The molecular target of benzodiazepine drugs are the receptors for the neurotransmitter GABA (GABA_{A}-receptor) which contain binding sites specific for benzodiazepines. Additionally, there are other binding sites on the GABA_{A} receptors, including the barbiturate and neurosteroid binding sites. GABA_{A}-receptors occur in various isoforms, distinguished mainly by the type of α-subunit, and are termed α1, α2, α3, and α5 GABA_{A}-receptor subtypes. The classical benzodiazepines such as diazepam interact with comparable affinity and efficacy with all benzodiazepine -sensitive GABA_{A} receptors. In addition to benzodiazepines, chemically unrelated ligands can likewise act at this site.

Thus, there is a need in the art for agents that can treat anxiety-related disorders without, or with a decreased severity of, side effects, such as dependence liability and sedation.

Citation or identification of any reference in Section 2 or in any other section of this application shall not be construed as an admission that such reference is available as prior art to the present invention.

### 3. SUMMARY OF THE INVENTION

The present invention is directed to a method for identifying a selective anxiolytic therapeutic agent that selectively or preferentially binds to the α2-GABA_{A} receptor as compared to the α1-GABA_{A} receptor, which agent allows for the treatment of an anxiety-related disorder while minimizing the unwanted side effects of such treatment mediated through the α1-GABA_{A} receptor. The method comprises contacting a candidate molecule (test agent) with the α2-GABA_{A} receptor and the α1-GABA_{A} receptor and determining whether the candidate molecule selectively or preferentially binds to the α2-GABA_{A} receptor as compared to the α1-GABA_{A} receptor. The present invention is also directed to a selective anxiolytic therapeutic agent which selectively or preferentially binds the α2-GABA_{A} receptor as compared to the α1-GABA_{A} receptor. The present invention is also directed to a method of treating an anxiety-related disorder comprising administering to a subject in need of such treatment a therapeutically effective amount of a selective anxiolytic therapeutic agent which selectively or preferentially binds to the α2-GABA_{A} receptor as compared to the α1-GABA_{A} receptor. A selective agent of the present invention can also have a lesser or stronger binding affinity for the α3-GABA_{A} receptor or the α5-GABA_{A} receptor, relative to the α2-GABA_{A} receptor.

The selective agent can bind to the GABA_{A} receptor at any binding site on the receptor, including the neurosteroid, barbiturate or the benzodiazepine binding site of the receptor. It is believed that as long as the selective anxiolytic agent selectively or preferentially binds to or activates the α2-GABA_{A} receptor as compared to the α1-GABA_{A} receptor, α3-GABA_{A} receptor, or α5-GABA_{A} receptor, the binding site of the receptor to which the selective agent binds is immaterial to the present invention.

The present invention is based, in part, on the fact that the anti-anxiety effect of anxiolytic therapeutic agents, such as benzodiazepines, are mediated through the α2-GABA_{A} receptor, whereas the side effects of such agents, such as sedation and dependence liability, are mediated through the α1-GABA_{A} receptor.

According to the present invention, a selective anxiolytic therapeutic agent is an agent which preferentially or selectively binds to the α2-GABA_{A} receptor., as compared to the α1-GABA_{A} receptor. For example, the benzodiazepine diazepam binds equally well (non-specifically) to the α2-GABA_{A} receptor and the α1-GABA_{A} receptor, and thus, is not a selective agent. However, a selective therapeutic agent either has less binding affinity to the α1-GABA_{A} receptor or has greater binding affinity to the α2-GABA_{A} receptor., as compared to a non-selective agent. In an embodiment of the present invention, selective or preferential binding indicates that the agent binds to (has an affinity for) the α2-GABA_{A} receptor at a level that is at least 10% greater than the agent has for the α1-GABA_{A} receptor. In another embodiment, the agent binds the α2-GABA_{A} receptor at least two-fold better than the α1-GABA_{A} receptor. In another embodiment, the agent binds the α2-GABA_{A} receptor at least ten-fold better than the α1-GABA_{A} receptor. In yet another embodiment, the agent binds the α2-GABA_{A} receptor at least one hundred-fold better than the α1-GABA_{A} receptor. In yet another embodiment, the agent binds the α2-GABA_{A} receptor at least one thousand fold better than the α1-GABA_{A} receptor. A selective agent of the present invention can also have a lesser or stronger binding affinity for the α3-GABA_{A} receptor or the α5-GABA_{A} receptor, relative to the α2-GABA_{A} receptor.

In an alternative embodiment of the present invention, a selective anxiolytic therapeutic agent is an agent which non-selectively binds the α2-GABA_{A} receptor, as compared to the α1-GABA_{A} receptor, yet the efficacy of receptor activation differs. For example, the binding affinity of the agent is not different between the two receptor sub-types but the ability of the agent to activate the α2-GABA_{A} receptor is greater than the ability of the agent to activate the α1-GABA_{A} receptor, thus the agent acts as a selective agent. In one aspect of this embodiment, the α2-GABA_{A} receptor is activated by at least 10% greater than the α1-GABA_{A} receptor. In another aspect of this embodiment, the α2-GABA_{A} receptor is activated by at least 25% greater than the α1-GABA_{A} receptor. In a preferred aspect of this embodiment, the α2-GABA_{A} receptor is activated by at least 10% greater than the α1-GABA_{A} receptor. In a more preferred aspect of this embodiment, the α2-GABA_{A} receptor is activated by at least 50% greater than the α1-GABA_{A} receptor. Thus, the present invention is also directed to a method for identifying a selective anxiolytic therapeutic agent that selectively or preferentially activates the α2-GABA_{A} receptor as compared to the α1-GABA_{A} receptor irrespective of its binding affinities for the α1-GABA_{A} or α2-GABA_{A} receptor, which agent allows for the treatment of an anxiety-related disorder while minimizing the unwanted side effects of such treatment mediated through the α1-GABA_{A} receptor. The method comprises contacting a candidate molecule (test agent) with the α2-GABA_{A} receptor and the α1-GABA_{A} receptor and determining whether the candidate molecule selectively or preferentially activates the α2-GABA_{A} receptor as compared to the α1-GABA_{A} receptor. The present invention is also directed to a selective anxiolytic therapeutic agent which selectively or preferentially activates the α2-GABA_{A} receptor as compared to the α1-GABA_{A} receptor. The present invention is also directed to a method of treating an anxiety-related disorder comprising administering to a subject in need of such treatment a therapeutically effective amount of a selective anxiolytic therapeutic agent which selectively or preferentially activates the α2-GABA_{A} receptor as compared to the α1-GABA_{A} receptor. A selective agent of the present invention can also have a greater or lesser ability to activate the α3-GABA_{A} receptor or the α5-GABA_{A} receptor, relative to the α2-GABA_{A} receptor.

The present invention is also directed to methods of identifying a molecule that decreases the ability of a non-selective benzodiazepine to bind to the α1-GABA_{A} receptor but does not substantially decrease the ability of the non-selective benzodiazepine to bind to the α2-GABA_{A} receptor. Such a molecule allows for the treatment of an anxiety-related disorder with a benzodiazepine but with decreased side effects. The method comprises contacting the α1-GABA_{A} receptor and the α2-GABA_{A} receptor with a benzodiazepine and a candidate molecule (test agent) and detecting the ability of the candidate molecule to decrease the ability of the benzodiazepine to bind to the α1-GABA_{A} receptor but not substantially decrease the ability of the benzodiazepine to bind to the α2-GABA_{A} receptor. In one aspect of this embodiment, the binding to the α2-GABA_{A} receptor is decreased by not more than 75% and the binding to the α1-GABA_{A} receptor is decreased by at least 25%. In a preferred aspect of this embodiment, the binding to the α2-GABA_{A} receptor is decreased by not more than 50% and the binding to the α1-GABA_{A} receptor is decreased by at least 50%. In another preferred aspect of this embodiment, the binding to the α2-GABA_{A} receptor is decreased by not more than 25% and the binding to the α1-GABA_{A} receptor is decreased by at least 75%.

The present invention is also directed to methods of identifying a molecule that decreases the ability of a non-selective benzodiazepine to activate the α1-GABA_{A} receptor, α3-GABA_{A} receptor, or α5-GABA_{A} receptor but does not substantially decrease the ability of the non-selective benzodiazepine to activate the α2-GABA_{A} receptor. Such a molecule allows for the treatment of an anxiety-related disorder with a benzodiazepine but with decreased side effects. The method comprises contacting the α1-GABA_{A} receptor and the α2-GABA_{A} receptor with a benzodiazepine and a candidate molecule (test agent) and detecting the ability of the candidate molecule to decrease the ability of the benzodiazepine to activate the α1-GABA_{A}, α3-GABA_{A} receptor or α5-GABA_{A} receptor but not substantially decrease the ability of the benzodiazepine to activate the α2-GABA_{A} receptor. In one aspect of this embodiment, the activation of the α2-GABA_{A} receptor is decreased by not more than 75% and the activation of the α1-GABA_{A} receptor, α3-GABA_{A} receptor, or α5-GABA_{A} receptor, is decreased by at least 25%. In a preferred aspect of this embodiment, the activation of the α2-GABA_{A} receptor is decreased by not more than 50% and the activation of the α1-GABA_{A} receptor, α3-GABA_{A} receptor, or α5-GABA_{A} receptor, is decreased by at least 50%. In another preferred aspect of this embodiment, the activation of the α2-GABA_{A} receptor is decreased by not more than 25% and the activation of the α1-GABA_{A} receptor, α3-GABA_{A} receptor, or α5-GABA_{A} receptor, is decreased by at least 75%.

In an alternative embodiment of the present invention, a selective anxiolytic therapeutic agent is an agent whose specificity for the α2-GABA_{A} receptor stems partially from a higher affinity for the α2-GABA_{A} receptor as compared to the α1-GABA_{A} receptor and partially from an increased efficacy of activation of the α2-GABA_{A} receptor as compared to the α1-GABA_{A} receptor. Thus, the present invention includes all possible permutations in affinity and efficacy which provides preferential activation of α2-GABA_{A} receptors as compared to α1-GABA_{A} receptors. The activation of α3- and α5-GABA_{A} receptors may vary independently from those of α1- and α2-GABA_{A} receptors in affinity or intrinsic activity.

The selective anxiolytic therapeutic agents and/or molecules of the present invention include pro-drugs that are metabolized in vivo to a biologically active agent, i.e., a selective anxiolytic agent or modulator of binding.

### 4. BRIEF DESCRIPTION OF THE FIGURES

FIGS. 1A-D. Targeting of the α2 subunit GABA_{A} receptor gene. FIG. 1A: Structure of wild type and mutant alleles. Mutant allele 1 is obtained after gene targeting in mouse ES cells and introduced into the mouse germline; breeding of these mice to Ella-cre mice results in excision of the neomycin resistance cassette (mutant allele 2). The 5′ and 3′ probes, which are flanking the targeting vector, are drawn as solid bars. His and Arg denote codons for histidine and arginine, respectively, at position 101 in exon 4. FIG. 1B: Southern blot analysis of wild type (wt) allele and mutant allele 1 (Mut1) in embryonic stem cells. FIG. 1C: Genotyping offspring from a cross of a chimera and a mouse hemizygous for the Ella-cre transgene. Top panel: PCR primers P1, P2 and P3 provide specific amplification products for each allele. Bottom panel: PCR primers UR26 and UR36 amplify the cre transgene. Fig. ID: Verification of the α2(H101R) point mutation by automated DNA sequencing.

FIGS. 2A-C. Molecular characteristics of GABA_{A} receptors in α2(H101R) and α3(H126R) mice. FIG. 2A: Western blots of whole brain membranes from wild type and α2(H101R) and α3(H126R) mice using antisera recognizing the α1, α2, α3, β2/3 and γ2 subunits. FIG. 2B: Receptor autoradiography of diazepam-insensitive sites in wild type, α2(H101R) (upper panel) and α3(H126R) brain (lower panel). Parasagittal sections were incubated with 10 nM [³H]Ro15-4513 in the presence of 100 μM diazepam to reveal the diazepam-insensitive [³H]Ro15-4513 binding sites. In wild type mice, diazepam-insensitive [³H]Ro15-4513 binding is due to α4 and α6 GABA_{A} receptors. FIG. 2C: GABA responses in cultured hippocampal pyramidal cells from α2(H101R) mice. The holding potential in the patch-clamp analysis was −60 mV the chloride concentration symmetrical. GABA was applied for 5 sec. Hippocampal neurons from E16.5 embryos were cultured for 10-14 days. Double asterisk, P<0.00 1 (Student's t-test).

FIGS. 3A-F. Behavioral assessment of the sedative, motor impairing and anticonvulsant properties of diazepam in α2(H101R) (FIGS. 3A-C) and α3(H126R) mice (FIGS. 3D-F) in comparison to wild type mice. FIG. 3A: Dose-dependent inhibition of locomotor activity in wild type and α2(H101R) mice [F(3,71) 19.31, P<0.001, n=9-10 mice per group]. FIG. 3B: Dose-dependent decrease in the latency to fall off the rotating rod (fixed 2 rpm) in wild type and α2(H101R) mice [F(3,153)=71.01, P<0.001, n=20-21 mice per group]. FIG. 3C: Dose-dependent decrease of the percentage of mice developing tonic convulsions in wild type [x²=32.38, P<0.001, n=10 mice per group] and α2(H101R) mice [x²=28.13, P<0.001, n=10 mice per group]. FIG. 3D: Dose-dependent inhibition of locomotor activity in wild type and α3(H126R) mice [F(3,64)=14.70, P<0.001,17=8-10 mice per group]. FIG. 3E: Dose-dependent decrease in the latency to fall off the rotating rod (fixed 2 rpm) in wild type and α3(H126R) mice [F(3,64)=36.78, F<0.001, n 8-10 mice per group]. FIG. 3F: Dose-dependent decrease of the percentage of mice developing tonic convulsions in wild type [x2=32.38, P<0.001, n=10 mice per group] and α3(H126R) mice [x2=28.60, P<0.001, n=10 mice per group]. Results are given as means ±S.E.M.+, P<0.05,++, P<0.01 and +++P<0.001 (Dunnett's post-hoc comparisons or Fisher's exact tests). V, vehicle. The rotarod and pentylenetetrazole convulsion tests were performed according to Bonetti et al., 1988, Pharmacol. Biochem. Behav. 31:733. Locomotor activity was automatically recorded for 30 min. Mice were treated with either vehicle or iazepam (3, 10 and 30 mg/kg p.o.) 30 minutes prior to testing.

FIGS. 4A-F. Behavioral assessment of anxiolytic-like action of diazepam in α2(H101R) (FIGS. 4A-C) and α3(H126R) mice (FIGS. 4D-F) in comparison to wild type mice. FIG. 4A: Light/dark choice test. Diazepam dose-dependently increased the time spent in the lit area in wild type mice [F(3,36)=3.14, P<0.05] but not in α2(H101R) mice [F(3,36)=0.32, n.s.], n=10 mice per group. FIGS. 4B-C: Elevated plus-maze. Diazepam (2 mg/kg) increased the percentage of time spent and entries on the open arms in wild type [P<0.01 and P<0.05 versus vehicle] but not in α2(H101R) mice [F(1,32)=4.31 and F(1,32)=4.76, P<0.05, respectively, n=8-10 per group]. FIG. 4D: Light/dark choice test. Dose-dependent increase in the time spent in the lit area in wild type and α3(H126R) mice [F(1,70)=14.74, P<0.001, n 9-10 mice per group]. FIGS. 4E-F: Elevated plus-maze. Diazepam (2 mg/kg) increased the percentage of time spent on the open arms and the umber of entries on the open arms to the same extent in wild type and α3(H126R) mice [F(1 ,36)=26.52 and F(1,36)=37.31, P<0.001, respectively, n=10 per group]. Results are given as means ±S.E.M.+, P<0.05;++, P<0.01;+++P<0.001 (Dunnett's or Fisher's pair-wise post-hoc comparisons or Fisher's exact tests). V, vehicle, Dz, diazepam. The light-dark choice test was carried out as described infra with an illumination of 500 lux. Mice were given vehicle or increasing doses of diazepam (0.5, 1 and 2 mg/kg p.o.). The elevated plus-maze was performed as described infra under an indirect dim light illumination (<10 lux). Vehicle or diazepam were administered p.o. 30 minutes prior to testing.

### 5. DETAILED DESCRIPTION OF THE INVENTION

The present invention is directed to a method for identifying a selective anxiolytic therapeutic agent that selectively or preferentially binds to the α2-GABA_{A} receptor as compared to the α1-GABA_{A} receptor, which agent allows for the treatment of an anxiety-related disorder while minimizing the unwanted side effects of such treatment mediated through the α1-GABA_{A} receptor. The method comprises contacting a candidate molecule (test agent) with the α2-GABA_{A} receptor and the α1-GABA_{A} receptor and determining whether the candidate molecule selectively or preferentially binds to the α2-GABA_{A} receptor as compared to the α1-GABA_{A} receptor. The present invention is also directed to a selective anxiolytic therapeutic agent which selectively or preferentially binds the α2-GABA_{A} receptor as compared to the α1-GABA_{A} receptor. The present invention is also directed to a method of treating an anxiety-related disorder comprising administering to a subject in need of such treatment a therapeutically effective amount of a selective anxiolytic therapeutic agent which selectively or preferentially binds to the α2-GABA_{A} receptor as compared to the α1-GABA_{A} receptor. A selective agent of the present invention can also have a lesser or stronger binding affinity for the α3-GABA_{A} receptor or the α5-GABA_{A} receptor, relative to the α2-GABA_{A} receptor.

The selective agent can bind to the GABA_{A} receptor at any binding site on the receptor, including the neurosteroid, barbiturate or the benzodiazepine binding site of the receptor. It is believed that as long as the selective anxiolytic agent selectively or preferentially binds to or activates the α2-GABA_{A} receptor as compared to the α1-GABA_{A} receptor, α3-GABA_{A} receptor, or α5-GABA_{A} receptor, the binding site of the receptor to which the selective agent binds is immaterial to the present invention.

The present invention is based, in part, on the fact that the anti-anxiety effect of anxiolytic therapeutic agents, such as benzodiazepines, are mediated through the α2-GABA_{A} receptor, whereas the side effects of such agents, such as sedation and dependence liability, are mediated through the α1-GABA_{A} receptor.

According to the present invention, a selective anxiolytic therapeutic agent is an agent which preferentially or selectively binds to the α2-GABA_{A} receptor, as compared to the α1-GABA_{A} receptor. For example, the benzodiazepine diazepam binds equally well (non-specifically) to the α2-GABA_{A} receptor and the α1-GABA_{A} receptor, and thus, is not a selective agent. However, a selective therapeutic agent either has less binding affinity to the α1-GABA_{A} receptor or has greater binding affinity to the α2-GABA_{A} receptor, as compared to a non-selective agent. In an embodiment of the present invention, selective or preferential binding indicates that the agent binds to (has an affinity for) the α2-GABA_{A} receptor at a level that is at least 10% greater than the agent has for the α1-GABA_{A} receptor. In another embodiment, the agent binds the α2-GABA_{A} receptor at least two-fold better than the α1-GABA_{A} receptor. In another embodiment, the agent binds the α2-GABA_{A} receptor at least ten-fold better than the α1-GABA_{A} receptor. In yet another embodiment, the agent binds the α2-GABA_{A} receptor at least one hundred-fold better than the α1-GABA_{A} receptor. In yet another embodiment, the agent binds the α2-GABA_{A} receptor at least one thousand fold better than the α1-GABA_{A} receptor. A selective agent of the present invention can also have a lesser or stronger binding affinity for the α3-GABA_{A} receptor or the α5-GABA_{A} receptor, relative to the α2-GABA_{A} receptor.

In an alternative embodiment of the present invention, a selective anxiolytic therapeutic agent is an agent which non-selectively binds the α2-GABA_{A} receptor, as compared to the α1-GABA_{A} receptor, yet the efficacy of receptor activation differs. For example, the binding affinity of the agent is not different between the two receptor subtypes but the ability of the agent to activate the α2-GABA_{A} receptor is greater than the ability of the agent to activate the α1-GABA_{A} receptor, thus the agent acts as a selective agent. In one aspect of this embodiment, the α2-GABA_{A} receptor is activated by at least 10% greater than the α1-GABA_{A} receptor. In another aspect of this embodiment, the α2-GABA_{A} receptor is activated by at least 25% greater than the α1-GABA_{A} receptor. In a preferred aspect of this embodiment, the α2-GABA_{A} receptor is activated by at least 10% greater than the α1-GABA_{A} receptor. In a more preferred aspect of this embodiment, the α2-GABA_{A} receptor is activated by at least 50% greater than the α1-GABA_{A} receptor. Thus, the present invention is also directed to a method for identifying a selective anxiolytic therapeutic agent that selectively or preferentially activates the α2-GABA_{A} receptor as compared to the α1-GABA_{A} receptor irrespective of its binding affinities for the α1-GABA_{A} or α2-GABA_{A} receptor, which agent allows for the treatment of an anxiety-related disorder while minimizing the unwanted side effects of such treatment mediated through the α1-GABA_{A} receptor. The method comprises contacting a candidate molecule (test agent) with the α2-GABA_{A} receptor and the α1-GABA_{A} receptor and determining whether the candidate molecule selectively or preferentially activates the α2-GABA_{A} receptor as compared to the α1-GABA_{A} receptor. The present invention is also directed to a selective anxiolytic therapeutic agent which selectively or preferentially activates the α2-GABA_{A} receptor as compared to the α1-GABA_{A} receptor. The present invention is also directed to a method of treating an anxiety-related disorder comprising administering to a subject in need of such treatment a therapeutically effective amount of a selective anxiolytic therapeutic agent which selectively or preferentially activates the α2-GABA_{A} receptor as compared to the α1-GABA_{A} receptor. A selective agent of the present invention can also have a greater or lesser ability to activate the α3-GABA_{A} receptor or the α5-GABA_{A} receptor, relative to the α2-GABA_{A} receptor.

The present invention is also directed to methods of identifying a molecule that decreases the ability of a non-selective benzodiazepine to bind to the α1-GABA_{A} receptor but does not substantially decrease the ability of the non-selective benzodiazepine to bind to the α2-GABA_{A} receptor. Such a molecule allows for the treatment of an anxiety-related disorder with a benzodiazepine but with decreased side effects. The method comprises contacting the α1-GABA_{A} receptor and the α2-GABA_{A} receptor with a benzodiazepine and a candidate molecule (test agent) and detecting the ability of the candidate molecule to decrease the ability of the benzodiazepine to bind to the α1-GABA_{A} receptor but not substantially decrease the ability of the benzodiazepine to bind to the α2-GABA_{A} receptor. In one aspect of this embodiment, the binding to the α2-GABA_{A} receptor is decreased by not more than 75% and the binding to the α1-GABA_{A} receptor is decreased by at least 25%. In a preferred aspect of this embodiment, the binding to the α2-GABA_{A} receptor is decreased by not more than 50% and the binding to the α1-GABA_{A} receptor is decreased by at least 50%. In another preferred aspect of this embodiment, the binding to the α2-GABA_{A} receptor is decreased by not more than 25% and the binding to the α1-GABA_{A} receptor is decreased by at least 75%.

The present invention is also directed to methods of identifying a molecule that decreases the ability of a non-selective benzodiazepine to activate the α1-GABA_{A} receptor but does not substantially decrease the ability of the non-selective benzodiazepine to activate the α2-GABA_{A} receptor. Such a molecule allows for the treatment of an anxiety-related disorder with a benzodiazepine but with decreased side effects. The method comprises contacting the α1-GABA_{A} receptor and the α2-GABA_{A} receptor with a benzodiazepine and a candidate molecule (test agent) and detecting the ability of the candidate molecule to decrease the ability of the benzodiazepine to activate the α1-GABA_{A} receptor but not substantially decrease the ability of the benzodiazepine to activate the α2-GABA_{A} receptor. In one aspect of this embodiment, the activation of the α2-GABA_{A} receptor is decreased by not more than 75% and the activation of the α1-GABA_{A} receptor is decreased by at least 25%. In a preferred aspect of this embodiment, the activation of the α2-GABA_{A} receptor is decreased by not more than 50% and the activation of the α1-GABA_{A} receptor is decreased by at least 50%. In another preferred aspect of this embodiment, the activation of the α2-GABA_{A} receptor is decreased by not more than 25% and the activation of the α1-GABA_{A} receptor is decreased by at least 75%.

5.1 Screening for Preferential Binding Agents

The present invention is directed to a method for identifying a selective anxiolytic therapeutic agent that selectively or preferentially binds to the α2-GABA_{A} receptor as compared to the α1-GABA_{A} receptor, which selective agent allows for the treatment of anxiety-related disorders while minimizing the side effects of such treatment which are mediated through the α1-GABA_{A} receptor. The method comprises contacting a candidate molecule with the α2-GABA_{A} receptor and the α1-GABA_{A} receptor and determining whether the candidate molecule selectively or preferentially binds to the α2-GABA_{A} receptor as compared to the α1-GABA_{A} receptor. In one embodiment of the present invention, the agent binds to (has an affinity for) the α2-GABA_{A} receptor at a level that is at least 10% greater than the agent has for the α1-GABA_{A} receptor. In another embodiment, the agent binds the α2-GABA_{A} receptor at least two-fold better than the α1-GABA_{A} receptor. In another embodiment, the agent binds the α2-GABA_{A} receptor at least ten-fold better than the α1-GABA_{A} receptor. In yet another embodiment, the agent binds the α2-GABA_{A} receptor at least one hundred-fold better than the α1-GABA_{A} receptor. In yet another embodiment, the agent binds the α2-GABA_{A} receptor at least one thousand fold better than the α1-GABA_{A} receptor.

Methods for screening that can be used to carry out the foregoing are commonly known in the art. See, e.g., the following references, which disclose screening of peptide libraries: Parmley and Smith, 1989, Adv. Exp. Med. Biol. 251:215-218; Scott and Smith, 1990, Science 249:386-390; Fowlkes et al., 1992, BioTechniques 13:422-427; Oldenburg et al., 1992, Proc. Natl. Acad. Sci. USA 89:5393—5397; Yu et al., 1994, Cell 76:933—945; Staudt et al., 1988, Science 241:577-580; Bock et al., 1992, Nature 355:56435 566; Tuerk et al., 1992, Proc. Natl. Acad. Sci. USA 89:6988—6992; Ellington et al., 1992, Nature 355:850-852; U.S. Pat. No. 5,096,815; U.S. Pat. No. 5,223,409; U.S. Pat. No. 5,198,346; Rebar and Pabo, 1993, Science 263:671-673; and International Patent Publication No. WO 94/18318.

In a specific embodiment, screening can be carried out by contacting the library members with an α1-GABA_{A} receptor and/or an α2-GABA_{A} receptor immobilized on a solid phase, and harvesting those library members that bind to the receptor. Examples of such screening methods, termed “panning” techniques, are described by way of example in Parmley and Smith, 1988, Gene 73:305-318; Fowlkes et al., 1992, BioTechniques 13:422-427; International Patent Publication No. WO 94/18318; and in references cited hereinabove.

In a preferred embodiment, both types of receptor are expressed on the surface of a cell and the cell is employed in the screening assays.

In another specific embodiment, candidate molecules are screened as competitive or non-competitive receptor ligands. In yet another specific embodiment, the binding assay used is the binding assay described in Section 6, infra.

Methods for screening may involve labeling the receptors with radioligands (e.g., ¹²⁵I or ³H), magnetic ligands (e.g., paramagnetic beads covalently attached to photobiotin acetate), fluorescent ligands (e.g., fluorescein or rhodamine) or enzyme ligands (e.g., luciferase or beta-galactosidase). The reactants that bind in solution can then be isolated by one of many techniques known in the art, including but not restricted to, co-immunoprecipitation of the labeled moiety using antisera against the unlabeled ligand (or a ligand labeled with a distinguishable marker from that used on the labeled moiety), immunoaffinity chromatography, size exclusion chromatography, and gradient density centrifugation.

Methods commonly known in the art are used to label the receptors or candidate molecules. Suitable labeling includes, but is not limited to, radiolabeling by incorporation of radiolabeled amino acids, e.g., ³H-leucine or ³⁵S-methionine, radiolabeling by post-translational iodination with ¹²⁵I or ¹³¹ I using the chloramine T method, Bolton-Hunter reagents, etc., labeling with ³²P using a kinase and inorganic radiolabeled phosphorous, biotin labeling with photobiotin-acetate and sunlamp exposure, etc. In cases where one of the receptor is immobilized the ligand is labeled. Where neither the receptor or candidate molecule is immobilized, each can be labeled with a distinguishable marker such that isolation of both moieties can be followed to provide for more accurate quantitation, and to distinguish the formation of binding complexes.

Typical binding conditions are, for example, but not by way of limitation, in an aqueous salt solution of 10-250 mM NaCl, 5-50 mM Tris-HCl, pH 5-8, and 0.5% Triton X-100 or other detergent that improves the specificity of interaction. Metal chelators and/or divalent cations may be added to improve binding and/or reduce proteolysis. Reaction temperatures may include 4, 10, 15, 22, 25, 35, or 42 degrees Celsius, and time of incubation is typically at least 15 seconds, but longer times are preferred to allow binding equilibrium to occur.

The physical parameters of complex formation can be analyzed by quantitation of complex formation using assay methods specific for the label used, e.g., liquid scintillation spectroscopy for radioactivity detection, enzyme activity measurements for enzyme labeling, etc. The reaction results are then analyzed utilizing Scatchard analysis, Hill analysis, and other methods commonly known in the art (see, e.g., Proteins, Structures, and Molecular Principles, 2^{nd} Edition (1993) Creighton, Ed., W. H. Freeman and Company, New York).

In a second common approach to binding assays, one of the binding species, i.e., receptor or ligand, is immobilized on a filter, in a microtiter plate well, in a test tube, to a chromatography matrix, etc., either covalently or non-covalently. Proteins can be covalently immobilized using any method well known in the art, for example, but not limited to the method of Kadonaga and Tjian (1986, Proc. Natl. Acad. Sci. USA 83:5889-5893, 1986), i.e., linkage to a cyanogen-bromide derivatized substrate such as CNBr-Sepahrose 4B. Where needed, the use of spacers can reduce steric hindrance by the substrate. Non-covalent attachment of proteins to a substrate include, but are not limited to, attachment of a protein to a charged surface, binding with specific antibodies, binding to a third unrelated interacting protein.

In specific embodiments, blocking agents to inhibit non-specific binding of reagents to other protein components, or absorptive losses of reagents to plastics, immobilization matrices, etc., are included in the assay mixture. Blocking agents include, but are not restricted to, bovine serum albumin, beta-casein, nonfat dried milk, Denhardt's reagent, Ficoll, polyvinylpyrolidine, nonionic detergents (NP40, Triton X-100, Tween 20, Tween 80, etc.), ionic detergents (e.g., SDS, LDS, etc.), polyethylene glycol, etc.

After binding is performed, unbound, labeled receptor is removed with the supernatant, and the immobilized receptor with any bound, labeled ligand is washed extensively. The amount of label bound is then quantitated using standard methods known in the art to detect the label.

5.2 Screening for Preferential Activators

The present invention is also directed to a method for identifying a selective anxiolytic therapeutic agent that selectively or preferentially activates the α2-GABA_{A} receptor as compared to the α1-GABA_{A} receptor irrespective of its binding affinities for the α1-GABA_{A} or α2-GABA_{A} receptor, which agent allows for the treatment of an anxiety-related disorder while minimizing the unwanted side effects of such treatment mediated through the α1-GABA_{A} receptor. The method comprises contacting a candidate molecule (test agent) with the α2-GABA_{A} receptor and the α1-GABA_{A} receptor and determining whether the candidate molecule selectively or preferentially activates the α2-GABA_{A} receptor as compared to the α1-GABA_{A} receptor. In one embodiment, the agent activates the α2-GABA_{A} receptor by at least 10% greater than the α1-GABA_{A} receptor. In another embodiment, the α2-GABA_{A} receptor is activated by at least 25% greater than the α1-GABA_{A} receptor. In a preferred embodiment, the α2-GABA_{A} receptor is activated by at least 10% greater than the α1-GABA_{A} receptor. In a more preferred embodiment, the α2GABA_{A} receptor is activated by at least 50% greater than the α1-GABA_{A} receptor.

Methods for screening that can carry out the foregoing are commonly known in the art. For example, and not by way of limitation, the ability of an agent to activate a particular receptor can be tested using cells in which the receptor is expressed. In an illustrative example, in which α2-GABA_{A} receptor activation is measured following its activation by GABA, cells expressing α2-GABA_{A} receptor are contacted with GABA and a candidate molecule or GABA alone and the amount of α2-GABA_{A} receptor activation is measured by a variety methods. Such methods include, but are not limited to, measuring electrophysiological changes in membrane potential or electrical currents; measuring biochemically changes in flow chloride ions using radioactive chloride ions (chloride flux assay); measuring changes in allosteric interaction between GABA and the receptor and/or the candidate molecule by radioligand binding using cell membranes (GABA shift assay).

5.3 Screening for Binding Modulators

The present invention is also directed to methods of identifying a molecule that decreases the ability of a non-selective benzodiazepine to bind to the α1-GABA_{A} receptor but does not substantially decrease the ability of the non-selective benzodiazepine to bind to the α2-GABA_{A} receptor. Such a molecule allows for the treatment of an anxiety-related disorder with a benzodiazepine but with decreased side effects. The method comprises contacting the α1-GABA_{A} receptor and the α2-GABA_{A} receptor with a benzodiazepine and a candidate molecule (test agent) and detecting the ability of the candidate molecule to decrease the ability of the benzodiazepine to bind to the α1-GABA_{A} receptor but not substantially decrease the ability of the benzodiazepine to bind to the α2-GABA_{A} receptor. In one aspect of this embodiment, the binding to the α2-GABA_{A} receptor is decreased by not more than 75% and the binding to the α1-GABA_{A} receptor is decreased by at least 25%. In a preferred aspect of this embodiment, the binding to the α2-GABA_{A} receptor is decreased by not more than 50% and the binding to the α1-GABA_{A} receptor is decreased by at least 50%. In another preferred aspect of this embodiment, the binding to the α2-GABA_{A} receptor is decreased by not more than 25% and the binding to the α1-GABA_{A} receptor is decreased by at least 75%.

The present invention is also directed to methods of identifying a molecule that decreases the ability of a non-selective benzodiazepine to activate the α1-GABA_{A} receptor but does not substantially decrease the ability of the non-selective benzodiazepine to activate the α2-GABA_{A} receptor. Such a molecule allows for the treatment of an anxiety-related disorder with a benzodiazepine but with decreased side effects. The method comprises contacting the α1-GABA_{A} receptor and the α2-GABA_{A} receptor with a benzodiazepine and a candidate molecule (test agent) and detecting the ability of the candidate molecule to decrease the ability of the benzodiazepine to activate the α1-GABA_{A} receptor but not substantially decrease the ability of the benzodiazepine to activate the α2-GABA_{A} receptor. In one aspect of this embodiment, the activation of the α2-GABA_{A} receptor is decreased by not more than 75% and the activation of the α1-GABA_{A} receptor is decreased by at least 25%. In a preferred aspect of this embodiment, the activation of the α2-GABA_{A} receptor is decreased by not more than 50% and the activation of the α1-GABA_{A} receptor is decreased by at least 50%. In another preferred aspect of this embodiment, the activation of the α2-GABA_{A} receptor is decreased by not more than 25% and the activation of the α1-GABA_{A} receptor is decreased by at least 75%.

The methods that can be used to carry out screening for such candidate molecules are commonly known in the art and/or those methods employed in Sections 5.1 and 5.2, supra and in Section 6, infra.

5.4 Candidate Molecules

Any molecule known in the art can be tested for its ability to preferentially or selectively bind to or activate the α2-GABA_{A} receptor or modulate the ability of a benzodiazepine or chemical derivative thereof to bind to the α1-GABA_{A} receptor. By way of example, binding can be detected by measuring binding of the candidate molecule to the α2-GABA_{A} receptor and to the α1-GABA_{A} receptor and determining if the molecule is preferentially or selectively binding to the α2-GABA_{A} receptor. In yet another example, receptor activation can be detected by measuring ability of the candidate molecule to activate the α2-GABA_{A} receptor and the α1-GABA_{A} receptor and determining if the molecule is preferentially or selectively activating the α2-GABA_{A} receptor. In certain embodiments, the candidate molecule can be directly provided to a cell expressing the α2-GABA_{A} receptor and the α1-GABA_{A} receptor or can be provided by providing their encoding nucleic acids under conditions in which the nucleic acids are recombinantly expressed to produce the candidate proteins within the α2-GABA_{A} and α1-GABA_{A} receptor expressing cell.

This embodiment of the invention is well suited to screen chemical libraries for molecules that preferentially or selectively bind the α2-GABA_{A} receptor as compared to the α1-GABA_{A} receptor. The chemical libraries can be peptide libraries, peptidomimetic libraries, chemically synthesized libraries, recombinant, e.g., phage display libraries, and in vitro translation-based libraries, other non-peptide synthetic organic libraries, etc.

Exemplary libraries are commercially available from several sources (ArQule, Tripos/PanLabs, ChemDesign, Pharmacopoeia). In some cases, these chemical libraries are generated using combinatorial strategies that encode the identity of each member of the library on a substrate to which the member compound is attached, thus allowing direct and immediate identification of a molecule that is an effective modulator. Thus, in many combinatorial approaches, the position on a plate of a compound specifies that compound's composition. Also, in one example, a single plate position may have from 1-20 chemicals that can be screened by administration to a well containing the interactions of interest. Thus, if modulation is detected, smaller and smaller pools of interacting pairs can be assayed for the modulation activity. By such methods, many candidate molecules can be screened.

Many diversity libraries suitable for use are known in the art and can be used to provide compounds to be tested according to the present invention. Alternatively, libraries can be constructed using standard methods. Chemical (synthetic) libraries, recombinant expression libraries, or polysome-based libraries are exemplary types of libraries that can be used.

The libraries can be constrained or semirigid (having some degree of structural rigidity), or linear or nonconstrained. The library can be a cDNA or genomic expression library, random peptide expression library or a chemically synthesized random peptide library, or non-peptide library. Expression libraries are introduced into the cells in which the assay occurs, where the nucleic acids of the library are expressed to produce their encoded proteins.

In one embodiment, peptide libraries that can be used in the present invention may be libraries that are chemically synthesized in vitro. Examples of such libraries are given in Houghten et al., 1991, Nature 354:84-86, which describes mixtures of free hexapeptides in which the first and second residues in each peptide were individually and specifically defined; Lam et al., 1991, Nature 354:82-84, which describes a “one bead, one peptide” approach in which a solid phase split synthesis scheme produced a library of peptides in which each bead in the collection had immobilized thereon a single, random sequence of amino acid residues; Medynski, 1994, Bio/Technology 12:709-710, which describes split synthesis and T-bag synthesis methods; and Gallop et al., 1994, J. Medicinal Chemistry 37(9):1233-1251. Simply by way of other examples, a combinatorial library may be prepared for use, according to the methods of Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; or Salmon et al., 1993, Proc. Natl. Acad. Sci. USA 90:11708-11712. PCT Publication No. WO 93/20242 and Brenner and Lerner, 1992, Proc. Natl. Acad. Sci. USA 89:5381-5383 describe “encoded combinatorial chemical libraries,” that contain oligonucleotide identifiers for each chemical polymer library member.

In a preferred embodiment, the library screened is a biological expression library that is a random peptide phage display library, where the random peptides are constrained (e.g., by virtue of having disulfide bonding).

Further, more general, structurally constrained, organic diversity (e.g., nonpeptide) libraries, can also be used. By way of example, a benzodiazepine library (see e.g., Bunin et al., 1994, Proc. Natl. Acad. Sci. USA 91:4708-4712) may be used.

Conformationally constrained libraries that can be used include but are not limited to those containing invariant cysteine residues which, in an oxidizing environment, cross-link by disulfide bonds to form cystines, modified peptides (e.g., incorporating fluorine, metals, isotopic labels, are phosphorylated, etc.), peptides containing one or more non-naturally occurring amino acids, non-peptide structures, and peptides containing a significant fraction of γ-carboxyglutamic acid.

Libraries of non-peptides, e.g., peptide derivatives (for example, that contain one or more non-naturally occurring amino acids) can also be used. One example of these are peptoid libraries (Simon et al., 1992, Proc. Natl. Acad. Sci. USA 89:9367-9371). Peptoids are polymers of non-natural amino acids that have naturally occurring side chains attached not to the alpha carbon but to the backbone amino nitrogen. Since peptoids are not easily degraded by human digestive enzymes, they are advantageously more easily adaptable to drug use. Another example of a library that can be used, in which the amide functionalities in peptides have been permethylated to generate a chemically transformed combinatorial library, is described by Ostresh et al., 1994, Proc. Natl. Acad. Sci. USA 91:11138-11142). Another illustrative example of a non-peptide library is a benzodiazepine library. See, e.g., Bunin et al., 1994, Proc. Natl. Acad. Sci. USA 91:4708-4712.

The members of the peptide libraries that can be screened according to the invention are not limited to containing the 20 naturally occurring amino acids. In particular, chemically synthesized libraries and polysome based libraries allow the use of amino acids in addition to the 20 naturally occurring amino acids (by their inclusion in the precursor pool of amino acids used in library production). In specific embodiments, the library members contain one or more non-natural or non-classical amino acids or cyclic peptides. Non-classical amino acids include but are not limited to the D-isomers of the common amino acids, α-amino isobutyric acid, 4-aminobutyric acid, Abu, 2-amino butyric acid; γ-Abu, ε-Ahx, 6-amino hexanoic acid; Aib, 2-amino isobutyric acid; 3-amino propionic acid; omithine; norleucine; norvaline, hydroxyproline, sarcosine, citrulline, cysteic acid, t-butylglycine, t-butylalanine, phenylglycine, cyclohexylalanine, B-alanine, designer amino acids such as β-methyl amino acids, Ca-methyl amino acids, Na-methyl amino acids, fluoro-amino acids and amino acid analogs in general. Furthermore, the amino acid can be D (dextrorotary) or L (levorotary).

In a specific embodiment, derivatives, fragments and/or analogs of benzodiazepines, especially peptidomimetics, are screened for activity preferential or selective binders of the α2-GABA_{A} receptor.

In another embodiment of the present invention, combinatorial chemistry can be used to identify agents that preferentially or selectively bind the α2-GABA_{A} receptor. Combinatorial chemistry is capable of creating libraries containing hundreds of thousands of compounds, many of which may be structurally similar. While high throughput screening programs are capable of screening these vast libraries for affinity for known targets, new approaches have been developed that achieve libraries of smaller dimension but which provide maximum chemical diversity. (See e.g., Matter, 1997, Journal of Medicinal Chemistry 40:1219-1229).

One method of combinatorial chemistry, affinity fingerprinting, has previously been used to test a discrete library of small molecules for binding affinities for a defined panel of proteins. The fingerprints obtained by the screen are used to predict the affinity of the individual library members for other proteins or receptors of interest (in the instant invention, the α1-GABA_{A} receptor or the α2-GABA_{A} receptor.) The fingerprints are compared with fingerprints obtained from other compounds known to react with the protein of interest to predict whether the library compound might similarly react. For example, rather than testing every ligand in a large library for interaction with the α1-GABA_{A} receptor, only those ligands having a fingerprint similar to other compounds known to have that activity could be tested. (See, e.g., Kauvar et al., 1995, Chemistry and Biology 2:107-118; Kauvar, 1995, Affinity fingerprinting, Pharmaceutical Manufacturing International. 8:25-28; and Kauvar, Toxic-Chemical Detection by Pattern Recognition in New Frontiers in Agrochemical Immunoassay, D. Kurtz. L. Stanker and J. H. Skerritt. Editors, 1995, AOAC: Wash. D.C., 305-312).

Kay et al., 1993, Gene 128:59-65 (Kay) discloses a method of constructing peptide libraries that encode peptides of totally random sequence that are longer than those of any prior conventional libraries. The libraries disclosed in Kay encode totally synthetic random peptides of greater than about 20 amino acids in length. Such libraries can be advantageously screened to identify the α1-GABA_{A} receptor modulators. (See also U.S. Pat. No. 5,498,538 dated Mar. 12, 1996; and PCT Publication No. WO 94/18318 dated Aug. 18, 1994).

A comprehensive review of various types of peptide libraries can be found in Gallop et al., 1994, J. Med. Chem. 37:1233-1251.

5.5 Therapeutics

The invention provides methods of treatment of anti-anxiety disorders by administration to a subject of an effective amount of a Therapeutic of the invention, including a selective anxiolytic agent or a modulator of α1-GABA_{A} receptor binding, in which unwanted side effects, such as dependence liability, are minimized. In a preferred aspect, the Therapeutic is substantially purified. The subject is preferably an animal, including but not limited to animals such as cows, pigs, horses, chickens, cats, dogs, etc., and is preferably a mammal, and most preferably human. In a specific embodiment, a nonhuman mammal is the subject. In another embodiment, the Therapeutic of the invention is a pro-drug which is metabolized in vivo to a selective anxiolytic agent or modulator of binding. Moreover, the Therapeutic is an active metabolite of the pro-drug or a finctional derivative or analog thereof.

The therapeutic selective agent can bind to the GABA_{A} receptor at any binding site on the receptor, including the neurosteroid, barbiturate or the benzodiazepine binding site of the receptor. It is believed that as long as the selective anxiolytic agent selectively or preferentially binds to or activates the α2-GABA_{A} receptor as compared to the α1-GABA_{A} receptor, α3-GABA_{A} receptor, or α5-GABA_{A} receptor, the binding site of the receptor to which the selective agent binds is immaterial to the present invention.

Various delivery systems are known and can be used to administer a Therapeutic of the invention, e.g., encapsulation in liposomes, microparticles, microcapsules, recombinant cells capable of expressing the Therapeutic, receptor-mediated endocytosis (see, e.g., Wu and Wu, 1987, J. Biol. Chem. 262:4429-4432), construction of a Therapeutic nucleic acid as part of a retroviral or other vector, etc. Methods of introduction include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

In another embodiment, the Therapeutic can be delivered in a vesicle, in particular a liposome (see Langer, 1990, Science 249:1527-1533, Treat et al., 1989, In: Liposomes in the Therapy of Infectious Disease and Cancer, Lopez-Berestein and Fidler (eds.), Liss, New York, pp. 353-365, Lopez-Berestein, ibid., pp. 317-327, see generally ibid.).

In yet another embodiment, the Therapeutic can be delivered in a controlled release system. In one embodiment, a pump may be used (see Langer, supra, Sefton, 1987, CRC Crit. Ref. Biomed. Eng. 14:201, Buchwald et al., 1980, Surgery 88:507, Saudek et al., 1989, N. Engl. J. Med. 321:574). In another embodiment, polymeric materials can be used (see Langer and Wise (eds.), 1979, Medical Applications of Controlled Release, CRC Pres., Boca Raton, Fla., Controlled Drug Bioavailability, Drug Product Design and Performance, Smolen and Ball (eds.), 1984, Wiley, N.Y., Ranger and Peppas, 1983, J. Macromol. Sci. Rev. Macromol. Chem. 23:61, see also Levy et al., Science 228:190, During et al., 1989, Ann. Neurol. 25:351, Howard et al., 1989, J. Neurosurg. 71:105). In yet another embodiment, a controlled release system can be placed in proximity of the therapeutic target, i.e., the brain, thus requiring only a fraction of the systemic dose (see, e.g., Goodson, 1984, In Medical Applications of Controlled Release, supra, Vol. 2, pp. 115-138). Other controlled release systems are discussed in the review by Langer, 1990, Science 249:1527-1533.

In a specific embodiment where the Therapeutic is a nucleic acid encoding a protein Therapeutic, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun, Biolistic, Dupont), or by coating with lipids or cell-surface receptors or transfecting agents, or by administering it in linkage to a homeobox-like peptide which is known to enter the nucleus (see, e.g., Joliot et al., 1991, Proc. Natl. Acad. Sci. USA 88:1864-1868), etc. Alternatively, a nucleic acid Therapeutic can be introduced intracellularly and incorporated within host cell DNA for expression, by homologous recombination.

The present invention also provides pharmaceutical compositions. Such compositions comprise a therapeutically effective amount of a Therapeutic, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the Therapeutic, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water or saline for injection can be provided so that the ingredients may be mixed prior to administration.

The Therapeutics of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the Therapeutic of the present invention which will be effective in the treatment of a particular disorder or condition, i.e., anxiety-related disorder, will depend on the nature of the disorder or condition, and can be determined by standard clinical techniques. In addition, in vitro assays optionally may be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to about 1 mg/kg body weight. Effective doses may be extrapolated from doseresponse curves derived from in vitro or animal model test systems.

Suppositories generally contain active ingredient in the range of about 0.5% to about 10% by weight; oral formulations preferably contain about 10% to about 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

5.6 Transgenic Animals

The present invention also provides non-human transgenic and nontransgenic animal models which have an inactivating mutation in the α2-GABA_{A} receptor or have an inactivating mutation in the α1-GABA_{A} receptor, α3-GABA_{A} receptor or a5 GABA_{A} receptor. In one embodiment, the α2 subunit of the GABA_{A} receptor is mutated at amino acid position 101, wherein His is replaced by Arg. In another embodiment, the α1 subunit of the GABA_{A} receptor is mutated at amino acid position 101, wherein His is replaced by Arg. In another embodiment, the α3 subunit of the GABA_{A} receptor is mutated at amino acid position 126, wherein His is replaced by Arg. In another embodiment, the α5 subunit of the GABA_{A} receptor is mutated at amino acid position 105, wherein His is replaced by Arg. In still another embodiment, the α3 subunit of the GABA_{A} receptor is inactivated by gene targeting (“gene knock-out”). Such animals can be generated by methods commonly known in the art, including those described in Section 6, infra. Other methods include employing recombinagenic oligonucleotides as described in U.S. Pat. No. 6,271,360.

The following series of examples are presented by way of illustration and not by way of limitation on the scope of the present invention.

### 6. EXAMPLE: DETERMINATION OF MOLECULAR AND NEURONAL SUBSTRATE FOR SELECTIVE ATTENUATION OF ANXIETY

Excessive or inappropriate anxiety can be controlled by enhancing GABAergic inhibitory neurotransmission using clinically effective benzodiazepine drugs (Shader and Greenblatt, 1993, New Engl. J. Med. 328(19):1398-1405). However, it has not been possible to date to pinpoint the GABA_{A} receptor subtype(s) which mediate(s) the attenuation of anxiety. Four types of diazepam-sensitive GABA_{A} receptors can be distinguished based on the presence of α1, α2, α3 or α5 subunits. The following experiments demonstrate that the receptor having the α2 subunits mediates the attenuation of anxiety.

6.1 Materials and Methods

6.1.1 Generation of α2(H101R) and α3(H126R) Mice

Genomic clones containing exons 4 and 5 of the GABA_{A} receptor α2 subunit gene (GABRA2) were obtained from a lambda phage library and a 6.0 kb genomic Pstl/Ncol fragment was chosen for inclusion as homologous DNA into the replacement targeting vector. The vector contained the desired point mutation in exon 4 and a loxP-flanked neomycin resistance cassette (RNA polymerase II promoter, bovine growth hormone polyadenylation signal) in intron 3, which was inserted into an engineered SaIl site in intron 3. The sequence TTT CAC AAT in exon 4 encoding the amino acids FHN (positions 100-1 02) was mutated to TTC CGG AAT encoding the amino acids FRN. The replacement vector was electroporated into embryonic stem cells (line E14) and correctly targeted clones were injected into C57BL6/J blastocysts. Mice carrying the mutant allele were crossed with Ella-cre mice on the 129/SvJ background to remove the neomycine resistance cassette from the germline by cre/loxP-mediated excision. Lakso et al.,1996, Proc. Natl. Acad. Sci. USA 93:5860. The cre transgene was subsequently bred out.

Starting from the chimeras, the mice were crossed for 6 generations onto the 129/SvJ background. Heterozygote crosses were set up to yield homozygous mutant, heterozygous and wild type mice. Typically, 20-25 breeding pairs of both homozygous mutant and wild type mice produced the experimental animals which were used at about 8-12 weeks of age. Each mouse was injected with diazepam only once.

A mouse genomic clone containing a 16.8 kb insert including exons 3 and 4 of the GABA_{A} receptor α3 subunit gene (GABRA3) was obtained from a lambda phage library. A 4.3 kb HincII-BgIII fragment including exon 4 was selected as homologous DNA for the targeting vector. A loxP-flanked neomycin resistance (neo) marker (RNA polymerase II promoter, bovine growth hormone polyadenylation signal) was placed in the Ncol site downstream of exon 4. The sequence TTC GAO AAT in axon 4 encoding the amino acids FHN (positions 125-127) was mutated to TTC CGG MT encoding the amino acids FRN. Furthermore, a silent mutation for use in restriction fragment length polymorphism analysis was introduced: GGG (threonine at position 150) was replaced by GGT so that a novel Kpnl site (GGTACC) was generated. The final targeting vector RK-1 contained ca. 1.3 kb of homology 5′ of the mutation, ca. 0.2 1st of homology between the mutation and the neo marker and ca. 2.8 kb of homology 3′ of the neo marker. 5′ of the homology, a herpes simplex virus thymidine kinase (TK) cassette was included for negative selection. Out of several thousand ES cell clones analyzed by Southern blotting with a flanking probe, only one clone which was derived from embryonic stem cell line mEMS32 displayed correct targeting. Simpson et al., 1997, Nat Genet. 16:19. This clone was injected into blastocysts and the resulting mice were crossed with Ella-cre mice on the 129/SvJ background to eliminate the neomycine resistance cassette. Lakso et al., 1996, Proc. Natl. Acad. Sci. USA 93:5860. The cre transgene was subsequently bred out. Starting from the chimeras, the mice were crossed for 5 generations onto the 129/SvJ background. The breeding and experimental schemes were similar to the ones described for the α2(H101R) mice, taking into account that the gene for the α3 subunit is located on the X chromosome.

6.1.2 Light/Dark Choice Test

In a chamber containing an interconnected dark and lit (500 lux) box, the behavior of wild-type and mutant mice was tested for 5 min following the first entry into the dark compartment. Mice were treated with vehicle or diazepam (0.5, 1 and 2 mg/kg p.o.) 30 minutes prior to testing. The time spent in the lit box was recorded.

6.1.3 Elevated Plus-Maze

Mice were placed on an elevated crossbar with two walled and two open arms under indirect dim illumination. Wild-type and mutant mice were treated with vehicle or diazepam (2 mg/kg p.o.) 30 minutes prior to testing. The time spent on the open arms and the number of open arm entries was recorded for 5 minutes.

6.1.4 Behavioral Tests

Female wild-type and mutant mice were raised in the test rooms and behaviorally tested during the dark period of the 1 2-hr light/dark cycle. Diazepam was administered orally (4-5 ml/kg) suspended in saline containing 0.3% Tween 80, while pentylenetetrazole was dissolved in saline and administered ip.

6.1.5 Statistics

Data were analyzed using one or two-way ANOVAs followed by post-hoc mean comparisons with Dunnett's tests or multiple t-tests, respectively. When the Bartlett's test for homogeneity of variances was significant, data of each genotype were analyzed separately using Kruskall-Wallis and Mann-Whitney tests. Dichotomic variables were analyzed by Chi square and Fisher's exact tests. Wilcoxon signed-ranks tests were used for within group comparisons.

6.1.6 Locomotor Activity

Locomotor activity was recorded 30 minutes after the administration of vehicle or diazepam (3, 10 and 30 mg/kg p.o.) in a familiar automated two-chamber apparatus for 30 minutes.

6.1.7 Pentylenetetrazole Test

Following pentylenetetrazole treatment (120 mg/kg ip), occurrence and/or latency to tonic seizures was recorded for a maximum of 10 minutes in wild type and mutant mice. Treatment with either vehicle or diazepam (3,10 and 30 mg/kg p.o.) preceded pentylenetrazole by 30 minutes. This test is used to assess dependence liability.

6.1.8 Rotarod

To assess motor impairment, mice were trained to remain on a rod rotating at a fixed speed of 2 rpm for at least 120 seconds. Bonetti et al., 1988, Pharmacol. Biochem. Behav. 31:733. The latency to fall off the rod was recorded up to 60 seconds before and 30 minutes after treatment with either vehicle or diazepam (3,10 and 30 mg/kg po.).

6.2 Results

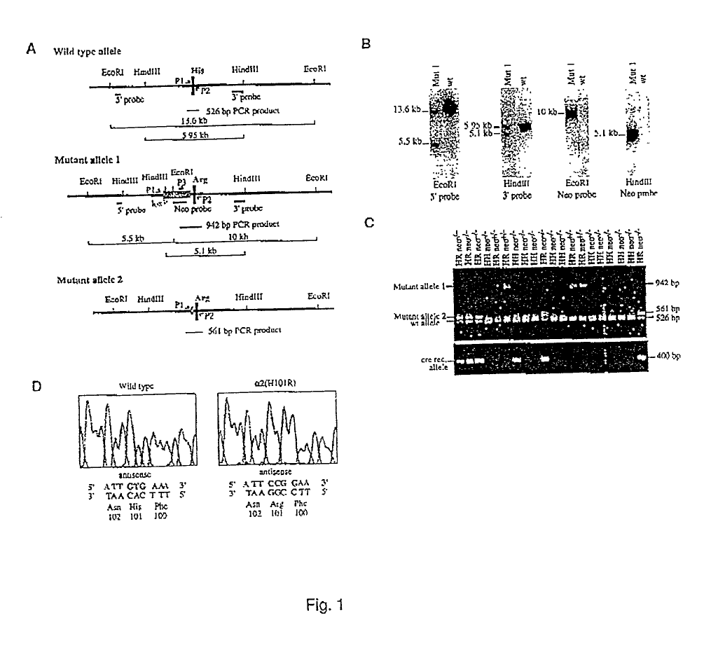

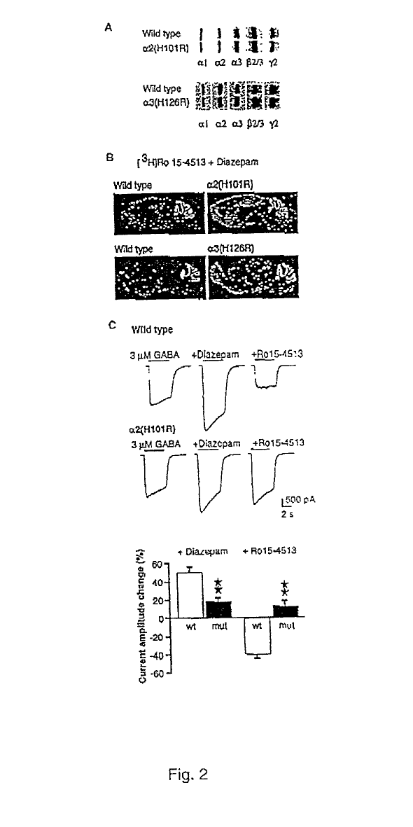

To distinguish between the pharmacology of α2 and α3 receptor subtypes, two mouse lines were generated in which a point mutation was introduced into homologous positions of the α2 subunit gene [α2(H101R)] (FIG. 1) and the α3-subunit gene [α3(H126R)], rendering the respective receptors diazepam-insensitive. The mutants did not display an overt distinctive phenotype, bred normally and expressed all subunits tested (α1, α2, α3, β2/3, γ2) at normal levels (FIG. 2A) and with unaltered distribution. The number of diazepam-insensitive [³H]Ro15-4513 (NEN, Boston, Mass.) binding sites was increased from 5% in wild type mice to 17% in α2(H101R) mice and to 11% in α3(H126R) mice [α2 wild type controls: Bₘₐₓ=0.08±0.02 pmol/mg protein, KD±4.3±0.8 nM (n=3); α2(H101R) mice: Bₘₐₓ=0.26±0.01 pmol/mg protein, K_{D}=8.0±1.3 nM) (n=3); α3 wild type controls: Bₘₐₓ=0.06±0.01 pmol/mg protein K_{D=}4.9±2.1 nM (n=3); α3(H126R) mice: Bₘₐₓ=0.11±0.02 pmol/mg protein, K_{D}=6.5±1.6 nM (n=3)]. In line with the known distribution of the α2 subunit (Fritschy and Mohler, 1995, J. Comp. Neurol. 359:154), diazepam-insensitive sites α2(H101R) mice were visualized in all regions expressing α2 GABA_{A} receptors as shown autoradiographically in parasagittal brain sections using 10 nM [³H]Ro15-4513 in the presence of 100 μM diazepam (FIG. 2B). Similarly, in α3(H126R) mice, the novel diazepam-insensitive [³H]Ro15-4513 binding sites displayed a distribution corresponding to that of the α3 subunit (FIG. 2B) (Fritschy and Mohler, 1995, J. Comp. Neurol. 359:154). Following immunoprecipitation with α2 or α3 subunit-specific antisera, a more than 1,000-fold decrease in the affinity for diazepam to the α2 and α3 subunits from the respective mutant mice was revealed by [³H]Ro15-4513 binding.

The electrophysiological response to GABA (3 μM) was indistinguishable in cultured hippocampal pyramidal cells from wild-type and α2(H101R) mice (FIG. 2C). However, the potentiation by diazepam (1μM) was reduced in cells from α2(H101R) mice compared to cells from wild type mice [17.6±4.5% (n=29) versus 48.1±7.9% (n=18), P =0.001] (FIG. 2C), the remaining potentiation presumably being due to GABA_{A} receptors other than α2. The inverse agonistic action of Ro15-4513 (1μM) in wild type cells was converted into an agonistic response in cells derived from α2(H101R) mice [−39±5.2% (n =13) versus 11.7±7.5% (n=23), P=0.003] (FIG. 2C). This is consistent with the switch in efficacy of Ro15-4513 from inverse agonism to agonism demonstrated for recombinant α2(H101R)β3γ2 receptors expressed in HEK-293 cells (Benson et al., 1998, FEBS Lett. 431:400).

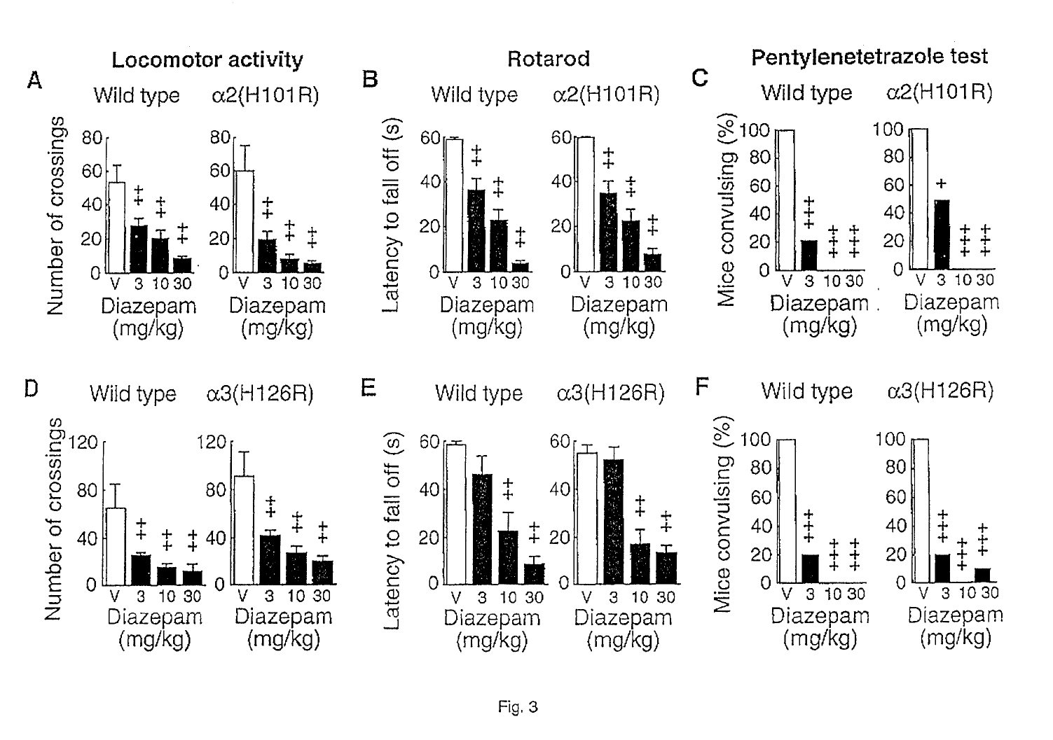

The pharmacological significance of the mutated α2 and α3 GABA_{A} receptors was assessed by comparing the diazepam-induced behavior of α2(H101R) and α3(H126R) mice with that of wild type mice. When tested in a dose-dependent manner, the sedative, motor impairing and anticonvulsant actions of diazepam (Bonetti et al., 1988, Pharmacol. Biochem. Behav. 31:733) were not impaired in either α2(H101R) mice or α3(H126R) mice as compared to wild type mice (FIG. 3).

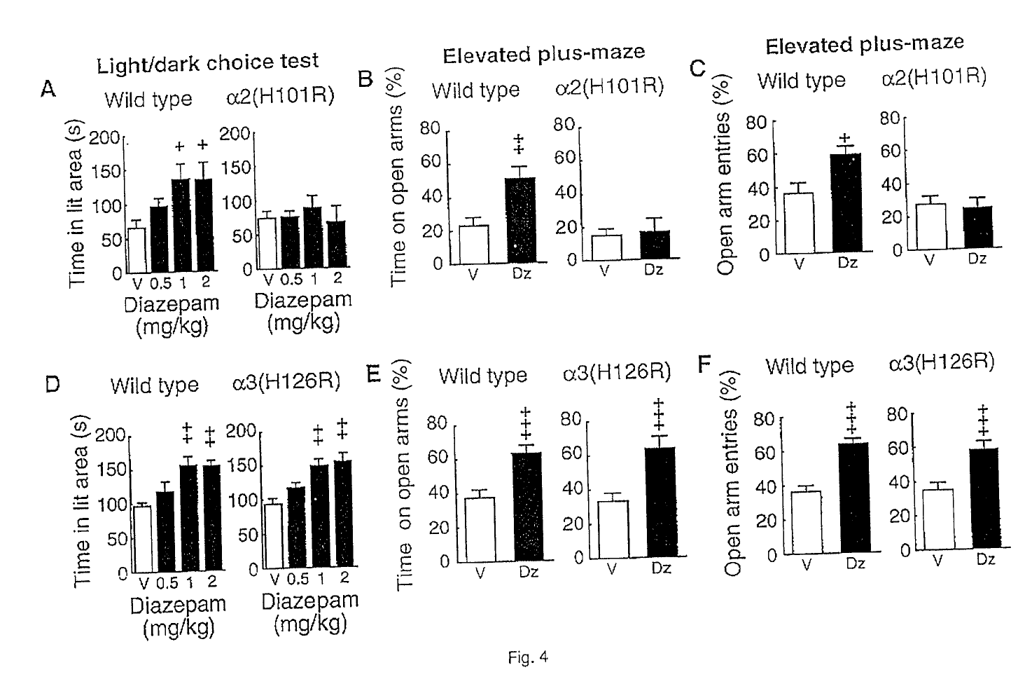

The anxiolytic-like action of diazepam in α2(H101R) and α3(H126R) mice was investigated in the light-dark choice test (Misslin et al., 1989, Behav. Proc. 18:119) and the elevated plus-maze test (Lister, 1987, Psychopharmacology 92:180). In the light-dark choice test, the α2(H101R) mice did not show the behavioral disinhibition by diazepam, which was apparent in wild type mice. Diazepam up to 2 mg/kg did not increase the time spent in the lit area in α2(H101R) mice in contrast to wild type mice (P<0.05 versus Vehicle) (FIG. 4A). This effect was not due to a motor deficit in α2(H101R) mice because no behavioral differences in the dark area were observed between wild type and α2(H101R) mice under either vehicle or diazepam treatment. Furthermore, α2(H101R) mice retained the ability to display an anxiolytic-like response to ligands acting at GABA_{A} receptor sites other than the benzodiazepine site. Sodium phenobarbital (15 mg/kg s.c.) induced a behavioral disinhibition in the light/dark choice test in α2(H101R) mice similar to that seen in wild type mice [time in the lit area, wt vehicle: 70.5=10.5 sec; wt phenobarbital; 111.75 =10.0 sec; α2(H101R) vehicle: 75.3=11.5 sec and α2(H101R) phenobarbital: 110.9=9.6 sec; F(1,24)=13.44, P<0.02, n=6-8]. The absence of an anxiolytic-like effect of diazepam in α2(H101R) mice was confirmed in the elevated plus-maze test. In wild type mice, diazepam facilitated the exploratory behavior by increasing both the amount of time spent [P<0.01 versus vehicle] and the number of entries in the open arms [P<0.05]. In contrast, in α2(H101R) mice diazepam failed to increase both parameters of exploratory behavior (FIG. 4B, C). Again, the failure was not due to motor impairment because the motor activity in the enclosed arms was similar in α2(H101R) and wild type mice irrespective of the treatment.

The potential contribution of α3 GABA_{A} receptors to the anxiolytic-like activity of diazepam was examined in α3(H126R) mice. Both α3(H126R) and wild type mice displayed similar dose-dependent anxiolytic-like responses to diazepam in the light/dark choice test [P<0.01 versus vehicle] (FIG. 4D) and in the elevated plus-maze [P<0.001 versus vehicle] (FIG. 4E,F). These results indicate that the anxiolytic action of diazepam in wild type mice does not involve interaction with α3 GABA_{A} receptors.

The anxiolytic-like action of diazepam is selectively mediated by the enhancement of GABAergic transmission in a population of neurons expressing the α2 GABA_{A} receptors, which represent only 15% of all diazepam-sensitive GABA_{A} receptors (Marksitzer et al., 1993, J. Recept. Res. 13:467). The α2 GABA_{A} receptor expressing cells in the cerebral cortex and hippocampus include pyramidal cells which display very high densities of α2 GABA_{A} receptors on the axon initial segment presumably controlling the output of these principal neurons (Nusser et al., 1998, Proc. Natl. Acad. Sci. USA 93:11939; Fritschy et al., 1998, J. Camp. Neurol. 390:194).

Regarding the reduction in side effects observed with chronic treatment, wild type mice which were chronically treated with diazepam exhibited withdrawal symptoms when administered flumazenil, a benzodiazepine antagonist. When mice containing a mutated α1-GABA_{A} receptor (H101R), which is insensitive to benzodiazepine, were subjected to the same protocol (chronic treatment with diazepam and administration of flumazenil), withdrawal symptoms, as measured by locomotor activity, were greatly reduced.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications and patent applications are cited herein, the disclosures of which are incorporated by reference in their entireties.

We claim:

1. A method for screening to identify a selective anxiolytic agent comprising contacting a candidate molecule with the α2-GABA_{A} receptor and the α1-GABA_{A} receptor and determining whether the candidate molecule selectively or preferentially binds to or activates the α2-GABA_{A} receptor as compared to the α1-GABA_{A} receptor, wherein a molecule that selectively or preferentially binds to or activates the α2-GABA_{A} receptor as compared to the α1-GABA_{A} receptor is a selective anxiolytic agent.

2. A method for screening to identify a selective anxiolytic agent comprising contacting a candidate molecule with the α2-GABA_{A} receptor and the α3-GABA_{A} receptor and determining whether the candidate molecule selectively or preferentially binds to or activates the α2-GABA_{A} receptor as compared to the α3-GABA_{A} receptor, wherein a molecule that selectively or preferentially binds to or activates the α2-GABA_{A} receptor as compared to the α3-GABA_{A} receptor is a selective anxiolytic agent.

3. A method for screening to identify a selective anxiolytic agent comprising contacting a candidate molecule with the α2-GABA_{A} receptor and the α5-GABA_{A} receptor and determining whether the candidate molecule selectively or preferentially binds to or activates the α2-GABA_{A} receptor as compared to the α5-GABA_{A} receptor, wherein a molecule that selectively or preferentially binds to or activates the α2-GABA_{A} receptor as compared to the α5-GABA_{A} receptor is a selective anxiolytic agent.

4. A selective anxiolytic agent which selectively or preferentially binds to or activates the α2-GABA_{A} receptor as compared to the α1-GABA_{A} receptor.

5. A selective anxiolytic agent which selectively or preferentially binds to or activates the α2-GABA_{A} receptor as compared to the α3-GABA_{A} receptor.

6. A selective anxiolytic agent which selectively or preferentially binds to or activates the α2-GABA_{A} receptor as compared to the α5-GABA_{A} receptor.

7. The selective anxiolytic agent according to claim 4, 5 or 6, wherein the agent binds to the benzodiazepine binding site of the receptor.

8. The selective anxiolytic agent according to claim 4, 5 or 6, wherein the agent binds to the neurosteroid binding site of the receptor.

9. The selective anxiolytic agent according to claim 4, 5 or 6, wherein the agent binds to the barbiturate binding site of the receptor.

10. A method of treating an anxiety-related disorder comprising administering a therapeutically effective amount of a selective anxiolytic agent and a pharmaceutically acceptable carrier to a patient in need thereof.

11. The method according to claim 10 in which the selective anxiolytic agent is identified by the method of claim 1, 2 or 3.

12. The method according to claim 10 in which the selective anxiolytic agent binds to the benzodiazepine binding site of the receptor.

13. The method according to claim 10 in which the selective anxiolytic agent binds to the neurosteroid binding site of the receptor.

14. The method according to claim 10 in which the selective anxiolytic agent binds to the barbiturate binding site of the receptor.

15. The method according to claim 10 in which the selective anxiolytic agent is a pro-drug.

16. A method of identifying a molecule that decreases the ability of a non-selective benzodiazepine to bind to the α1-GABA_{A} receptor but does not substantially decrease the ability of the non-selective benzodiazepine to bind to the α2-GABA_{A} receptor comprising contacting the α1-GABA_{A} receptor and the α2-GABA_{A} receptor with a non-selective benzodiazepine and a candidate molecule and detecting the ability of the candidate molecule to decrease the ability of the benzodiazepine to bind to the α1-GABA_{A} receptor but not substantially decrease the ability of the benzodiazepine to bind to the α2-GABA_{A} receptor.

17. A method of identifying a molecule that decreases the ability of a non-selective benzodiazepine to bind to the α3-GABA_{A} receptor but does not substantially decrease the ability of the non-selective benzodiazepine to bind to the α2-GABA_{A} receptor comprising contacting the α3-GABA_{A} receptor and the α2-GABA_{A} receptor with a non-selective benzodiazepine and a candidate molecule and detecting the ability of the candidate molecule to decrease the ability of the benzodiazepine to bind to the α3-GABA_{A} receptor but not substantially decrease the ability of the benzodiazepine to bind to the α2-GABA_{A} receptor.

18. A method of identifying a molecule that decreases the ability of a non-selective benzodiazepine to bind to the α5-GABA_{A} receptor but does not substantially decrease the ability of the non-selective benzodiazepine to bind to the α2-GABA_{A} receptor comprising contacting the α5-GABA_{A} receptor and the α2-GABA_{A} receptor with a non-selective benzodiazepine and a candidate molecule and detecting the ability of the candidate molecule to decrease the ability of the benzodiazepine to bind to the α5-GABA_{A} receptor but not substantially decrease the ability of the benzodiazepine to bind to the α2-GABA_{A} receptor.
